(19) United States
(12) Patent Application Publication
(10) Pub. No.: US 2009/0222011 A1
(43) Pub. Date: Sep. 3, 2009

(54) Targeting surgical instrument for use in spinal disc replacement and methods for use in spinal disc replacement

(75) Inventors: Jean Charles Lehuec, Pessac (FR); Loic Josse, Denens (CH); Mingyan Liu, Bourg la Reine (FR); Lukas Eisermann, San Diego, CA (US); Tai Friesem, Stockton On Tees (GB); Hallett H. Mathews, Williamsburg, VA (US); Thomas Zdeblick, Middleton, WI (US); Jeffrey Zhang, Collierville, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/038,977

(22) Filed: Feb. 28, 2008

(51) Int. Cl. A61F 5/00; A61B 17/04

(52) U.S. Cl. 606/87; 606/329; 606/330

(57) ABSTRACT

A targeting surgical instrument for use in spinal disc replacement includes a first arm, a second arm and a body portion. The first arm is configured to be longitudinally aligned with a mid-line of a spine and to be at least partially received on an endplate of a lower vertebra of spinal cavity. The spinal cavity is defined by a lower vertebra and an upper vertebra of the spinal cavity. The second arm is positioned at an angle relative to the first arm. The second arm defines an insertion angle for a spinal implant. The body portion connects the first arm and the second arm. The body portion includes a plurality of radial opaque markers located to allow alignment of the first arm on the mid-line of the spine.

### TECHNICAL FIELD

The present invention relates generally to the field of surgery and medical implants, and more particularly, to surgical tools and methods for use in positioning an intervertebral device between vertebral members of a patient.

### BACKGROUND OF THE INVENTION

The human spine is a biomechanical structure with thirty-three vertebral members, and is responsible for protecting the spinal cord, nerve roots and internal organs of the thorax and abdomen. The spine also provides structure support for the body while permitting flexibility of motion. A significant portion of the population will experience back pain at some point in their lives resulting from a spinal condition. The pain may range from general discomfort to disabling pain that immobilizes the individual. Back pain may result from a trauma to the spine, be caused by the natural aging process, or may be the result of a degenerative disease or condition.

Procedures to remedy back problems sometimes require correcting the distance between vertebral members by inserting an intervertebral device (e.g., spacer) between the members. The spacer, which is carefully positioned within the disc space and aligned relative to the vertebral members, is sized to position the vertebral members in a manner to alleviate the patient's back pain.

Further, the intervertebral device is preferably designed to facilitate insertion into a patient. That is, the shape and size of the device are designed to provide for minimal intrusion to a patient during insertion, but still be effective post-insertion to alleviate the pain and provide maximum mobility to the patient.

Major blood vessels are located at an anterior aspect of the spine and often the intervertebral device is designed to be inserted in the spinal cavity at the mid-line of the spine from an anterior aspect. Such an approach requires particular care relative to the blood vessels and/or other sensitive objects located at the spine mid-line when approaching the spinal cavity from the anterior direction.

Thus, a need exists for instruments and methods for inserting an intervertebral device into a spinal cavity which minimizes the opportunities for injuring blood vessels and/or other sensitive bodies in the vicinity of a mid-line of a spine.

### SUMMARY OF THE INVENTION

The present invention provides, in an aspect, a targeting surgical instrument for use in spinal disc replacement which includes a first arm, a second arm, and a body portion. The first arm is configured to be longitudinally aligned with a mid-line of a spine and to be at least partially received on an endplate of a lower vertebra of spinal cavity. The spinal cavity is defined by the lower vertebra and upper vertebra of the spinal cavity. The second arm is positioned at an angle relative to the first arm. The second arm defines an insertion angle. The body portion connects the first arm and the second arm. The body portion includes a plurality of radiopaque markers, which may be located to allow alignment of the first arm on the mid-line of the spine.

The present invention provides, in another aspect, a method for use in spinal disc replacement which includes aligning a first arm of a targeting instrument with a mid-line of a spine and placing the first arm on an endplate of a lower vertebra of a spinal cavity. The spinal cavity is defined by the lower vertebra and an upper vertebra of the spinal cavity. The targeting instrument includes a second arm aligned at an angle relative to the first arm and a body portion connecting the first arm and the second arm. The second arm defines an insertion angle for a spinal implant. The body portion includes a plurality of radiopaque markers, which may be located to allow alignment of the first arm on the mid-line of the spine.

### BRIEF DESCRIPTION OF THE DRAWINGS

The subject matter, which is regarded as the invention, is particularly pointed out and distinctly claimed in the claims at the conclusion of the specification. The foregoing and other objects, features, and advantages of the invention will be apparent from the following detailed description of preferred embodiments taken in conjunction with the accompanying drawings in which:

FIG. 1 is a perspective view of one embodiment of a targeting surgical instrument located on an endplate of a spinal cavity, in accordance with an aspect of the present invention;

FIG. 2 is a top elevational view of the targeting surgical instrument of FIG. 1, in accordance with an aspect of the present invention;

FIG. 3 is a side elevational view of the targeting surgical instrument of FIG. 1, in accordance with an aspect of the present invention;

FIG. 4 is a side elevational view of another embodiment of a targeting surgical instrument, in accordance with an aspect of the present invention;

FIG. 5 is a perspective view of the targeting surgical instrument and spinal cavity of FIG. 1 along with blood vessels near the spinal cavity, in accordance with an aspect of the present invention;

FIG. 6 is a side elevational view of the targeting surgical instrument and spinal cavity of FIG. 1, in accordance with an aspect of the present invention; and

FIG. 7 is a side elevational view of the targeting surgical instrument of FIG. 4 located in the spinal cavity of FIG. 1, in accordance with an aspect of the present invention;

### BEST MODE FOR CARRYING OUT THE INVENTION

In accordance with the principles of the present invention, a targeting surgical instrument for use in spinal disc replacement, and methods for use in implanting a prosthetic disc in a spinal cavity, are provided.

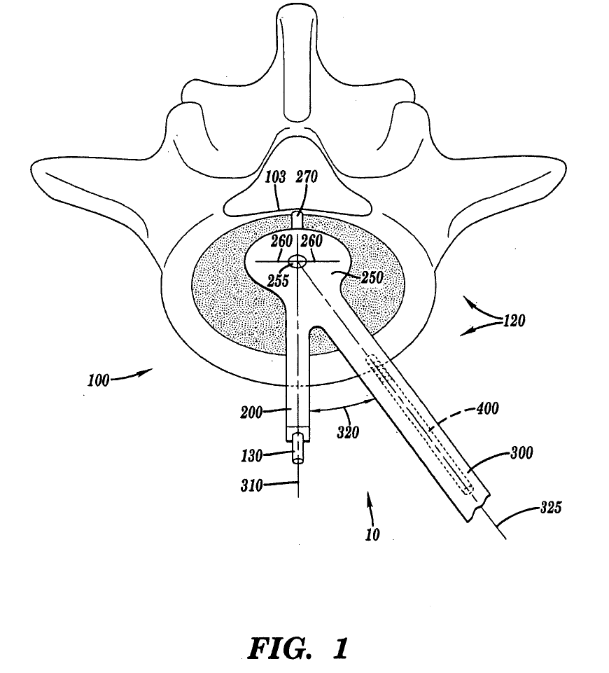

As depicted in FIG. 1, a targeting surgical instrument or tool 10 may be received on an endplate 110 of a lower (i.e., inferior) vertebra 100 defining a lower extent of a spinal cavity 120 into which it is desired to insert a spinal implant (not shown), e.g., a prosthetic disc. Such cavity may be created by a previous descectomy, i.e. the removal of a diseased or otherwise nonfunctional disc of a spinal column.

Tool 10 includes a first arm 200 and a second arm 300, which may be separated from, and positioned at an angle 320 relative to, each other. Angle 320 may be an acute angle, for example, an angle between about 10 degrees and about 45 degrees, such as about 35 degrees. First arm 200 and second arm 300 may be connected at a connecting body 250. Connecting body 250 may be rounded except for portions of an outer radial portion where first arm 200 and second arm 300 intersect connecting body 250 as depicted in FIG. 1. An intersection point of the longitudinal axis of first arm 200 (i.e., a first longitudinal axis 225) and second arm 300 (i.e., a second arm longitudinal axis 325) at a radial center 255 of connecting body 250 may be a rotation center (not shown) for a prosthesis (not shown) desired to be inserted in the spinal cavity. Tool 10 is particularly suited for inserting a prosthesis that is sensitive to the positioning accuracy of the rotation center on a mid-line of the spine. Connecting body 250 may include radiopaque markers 260 which may be located equidistant from a longitudinal axis 225 of first arm 200. Connecting body 250 may also include a radiopaque probe 270 located at a distal extent of connecting body 250 opposite an intersection point between first arm 200 and connecting body 250. Tool 10 may be formed of biocompatible plastic material (e.g., DELRINE). Also, as noted above, markers 260 and probe 270 may be made of radiopaque material, and the remainder of tool 10 may be formed of radio transparent material. Further, markers 260 and probe 270 may be formed integral to, or connected to, the remainder of connecting body 250. Markers 260 and/or probe 270 may be metal portions press fit into pre-cut grooves in the remainder of connecting body 250. Alternatively, markers 260 and/or probe 270 may be metal or other radiopaque portions, which are connected to, and/or extend from, connecting body 250.

After a disc (not shown) has been removed from a spine of a patient to form spinal cavity 120, first arm 200 may be aligned with a mid-line 310 of a spine or endplate (e.g., endplate 110) thereof as depicted in FIG. 1. More particularly, first arm 200 may be attached to a mid-line pin 130 previously attached (e.g., via impaction of the pin 130 into the endplate) to lower vertebra 100. As depicted in FIG. 5, first arm 200 may be attached to the mid-line pin by receiving the mid-line pin in a slot 202 of two extending portions 205 which extend from first arm 200 outside of the spinal cavity along an outer surface 105 of lower vertebra 100. These two extending portions 205 may be similar to some extent to the arms of a fork between which pin 130 is inserted. Alternatively, first arm 200 may be attached, or aligned with, mid-line 310 by a variety of other means. For example, an opening for receiving the pin surrounded on all sides by a descending (e.g., orthogonal to endplate 110) portion of arm 200 could be substituted for the slot.

Mid-line 310 of the spine may be identified intra-operatively using an anterior-posterior X-ray image. Mid-line pin 130 then may be attached (e.g, via impaction) to an outer surface 105 of lower vertebra 100. Also, a lateral X-ray image may be taken of the spinal cavity with tool 10 inserted therein (i.e., after first arm 200 is attached to mid-line pin 130). The lateral X-ray image may allow markers 260 and probe 270 to be visible relative to the remainder of tool 10. Thus, a distal extent of probe 270 may be adjusted by the user (e.g., a surgeon) such that probe 270 extends to, or near, a distal end 103 of endplate 100 without extending past such distal end. Markers 260 may be aligned such that an imaginary line connecting them is substantially orthogonal to mid-line 310 of the spinal column and contained in the plane defined by the two arms of the tool 10. More particularly, the markers may be aligned with one another (i.e., one behind the other) when viewed via the lateral X-ray image of the spinal cavity with tool 10 inserted therein. Such alignment may thereby locate longitudinal axis 225 of first arm 200 on mid-line 310 of the spine.

In another example, probe 270 may include a hook 280 extending distally from connecting body 250 and being curved to a position substantially orthogonal to a longitudinal axis of first arm 200 as depicted in FIG. 7. The hook may extend over the posterior edge of the endplate and may be manipulated to contact a distal side 107 of the lower vertebra and/or a posterior ligament (not shown). For example, hook 280 may be configured (e.g., shaped or dimensioned) to conform to a shape of endplate 110, distal side 107 of lower vertebra 100 and/or an intersection between endplate 110 and distal side 107.

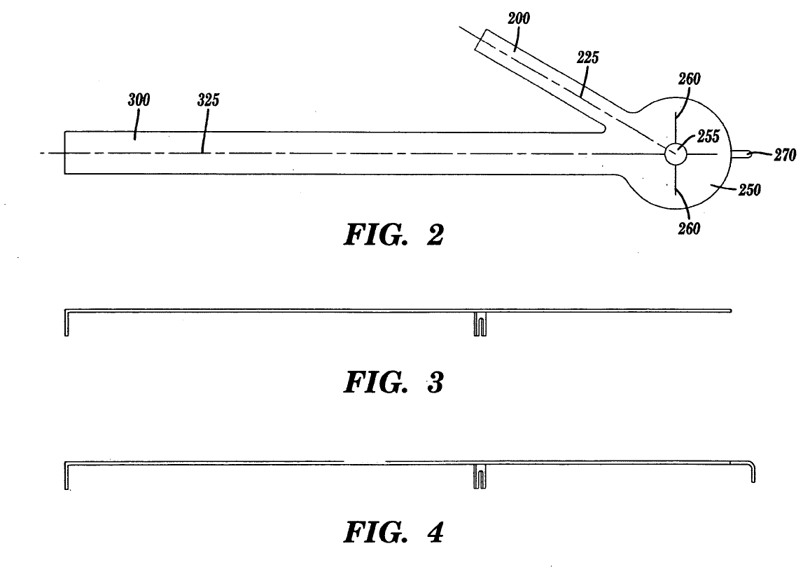

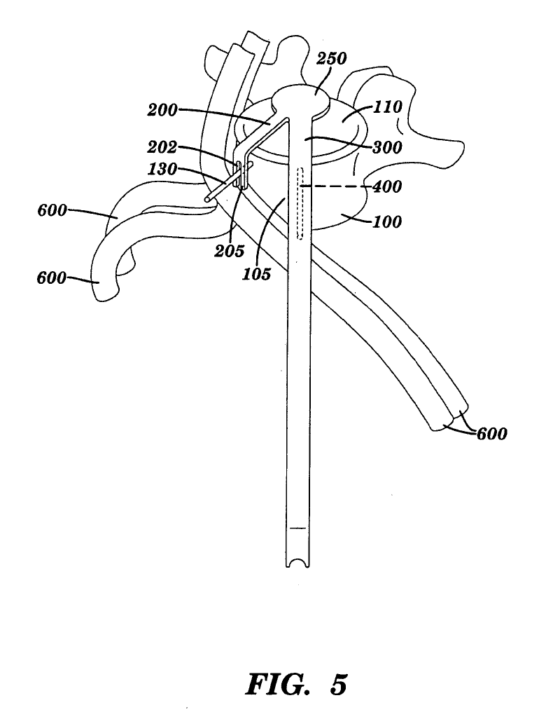

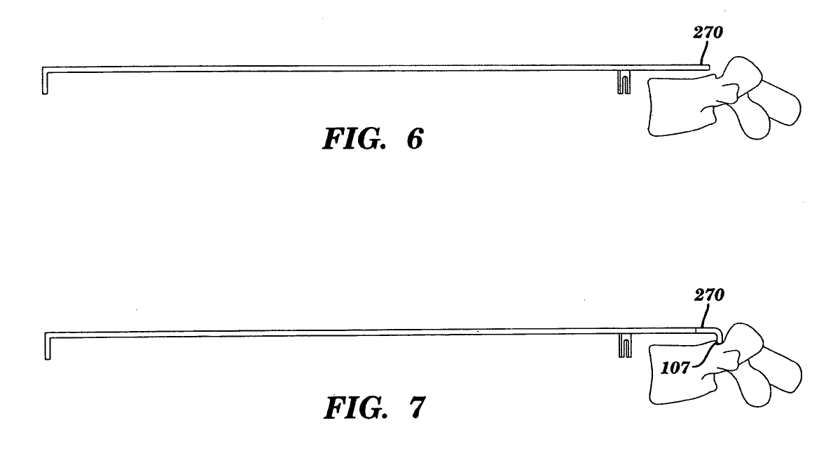

After longitudinal axis 225 of first arm 200 is aligned on the mid-line 310 of the spinal column, as depicted in FIG. 1, tool 10 may be held in place by a spreader (distraction tool) or other surgical tool (e.g., the tool which is the subject of co-owned U.S. patent application Ser. No. 11/344,946, filed Jan. 31, 2006, and entitled “A Spinal Disc Replacement Surgical Instrument And Methods For Use In Spinal Disc Replacement” (Attorney Docket No. P23436.00), and/or the tool which is the subject of co-owned U.S. patent application Ser. No. 10/768,354, filed Jan. 30, 2004 and entitled “Instrumentation and Methods for Preparation of an Intervertebral Space”, U.S. Publication No. US 2005/0113842A1, the entities of which are incorporated herein by reference). A targeting pin 400 (FIG. 1) may be placed (e.g., by impaction) along longitudinal axis 325 of second arm 300 on outer surface 105 of lower vertebra 100 as depicted in FIGS. 1-2. The second arm 300 may be longer than the first arm 200 because it is intended to be partially outside of the patient body. Targeting pin 400 may then be used as an indicator for a direction when inserting a surgical tool or marking the vertebra with a marker to allow preparation of the spinal cavity for receiving a spinal implant at an angle offset (e.g., at the angle of second arm 300) from mid-line 310 of the spine. For example, a keel cutter (not shown) may be utilized to cut channels or keels in an upper (not shown) and lower vertebra 100 as described for example in the co-owned patent applications indicated above. Such keels or channels are utilized to receive protruding portions (not shown) of a spinal implant (not shown). For example, an upper channel may be cut in the upper vertebra while tool 10 is located on endplate 110. Tool 10 may then be removed and the upper channel and targeting pin 400 may be utilized to align and cut a lower channel in endplate 110 of lower vertebra 100. Comers of the vertebra may also be cut to facilitate the insertion of the implant.

The channels may be aligned relative to a longitudinal axis of first arm 200 and therefore the mid-line 310 of the spine such that a central rotation point of an implant is located at the mid-line 310 of the spine, and at a location defined by radial center 255 of contacting body 250 of tool 10, when the implant is inserted in spinal cavity 120 with top and bottom protruding portions (not shown) of the implant being received in the channels (not shown) previously cut and a leading edge of the implant reaches the posterior wall (e.g., a radial end or edge of endplate 110) of the vertebra. As noted above, the insertion of the implant along the direction defined by targeting pin 400 and second arm 300 allows the implant and the tools used to facilitate the preparation of spinal cavity 120 for the implant to avoid contacting and/or damaging sensitive blood vessels 600 (FIG. 5) located at or near mid-line 310, and at the anterior side of the spine.

As will be understood by one skilled in the art, a surgical targeting instrument (e.g., tool 10) could be formed of a variety of materials and formed in a variety of shapes which are configured to be received in a spinal cavity and be aligned with a mid-line of a spine and provide an arm at an off set angle relative to the mid-line of the spine, which may provide a entry angle for a variety of surgical instruments which avoid contacting blood vessels 600 (FIG. 5) or other sensitive objects in the vicinity of the mid-line of the spine, and which prepare the spinal cavity for the implantation of an intervertebral device, e.g., a spinal implant or prosthetic. Further, such a surgical targeting instrument (e.g., tool 10) could include a variety of means for being aligned with a mid-line of a spine. For example, radial opaque markers and/or probes may be located at various locations to allow an appropriate aligning of the instrument within the spinal cavity.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

1. A targeting surgical instrument for use during a spinal disc replacement procedure, the instrument comprising:
a first arm configured to be longitudinally aligned with a mid-line of a spine and to be at least partially received on an endplate of a lower vertebra of a spinal cavity, the spinal cavity defined by the lower vertebra and an upper vertebra of the spinal cavity;
a second arm positioned at an angle relative to said first arm, said second arm defining an insertion angle for a spinal implant; and
a body portion connecting said first arm and said second arm, said body portion comprising a plurality of radiopaque markers.

2. The surgical instrument of claim 1, wherein said plurality of radiopaque markers is aligned with each other such that a line connecting said plurality of markers is substantially orthogonal to a longitudinal axis of said first arm.

3. The surgical instrument of claim 1, wherein said plurality of radiopaque markers is located equidistant from a centerline axis of said first arm.

4. The surgical instrument of claim 1, wherein said first arm is attachable to a pin attached to one of the lower vertebra and the upper vertebra.

5. The surgical instrument of claim 1, wherein said body portion further comprises a radiopaque probe at a distal end of said body opposite an interface between said body portion and said first arm.

6. The surgical instrument of claim 1, wherein said body portion further comprises a hook at a distal end of said body opposite an interface between said body portion and said first arm, said hook configured to extend to a posterior ligament to allow the placement of the body portion.

7. The surgical instrument of claim 1, wherein the insertion angle comprises an acute angle.

8. The surgical instrument of claim 7, wherein the acute angle comprises about 35 degrees.

9. A method for use in spinal disc replacement comprising:
aligning a first arm of a targeting instrument with a mid-line of a spine and placing the first arm on an endplate of a lower vertebra of a spinal cavity, the spinal cavity defined by the lower vertebra and an upper vertebra of the spinal cavity;
the targeting instrument comprising a second arm aligned at an angle relative to the first arm and a body portion connecting the first arm and the second arm, the second arm defining an insertion angle for a spinal implant; and
the body portion comprising a plurality of radiopaque.

10. The method of claim 9, further comprising placing a mid-line pin on the bottom vertebra at a mid-line of the spine and attaching the first arm to the pin.

11. The method of claim 9, further comprising aligning the markers with each other via a lateral X-ray image such that a longitudinal axis of the first arm is aligned with the mid-line of the spine.

12. The method of claim 9, further comprising aligning the markers with each other on the body portion such that a line connecting the plurality of markers is substantially orthogonal to a mid-line of the spine.

13. The method of claim 9, further comprising aligning the markers such that the plurality of radiopaque markers is located equidistant from a mid-line of the spine.

14. The method of claim 9, wherein the body portion further comprises a probe at a distal end of the body opposite an interface between the body portion and the first arm and further comprising adjusting a distal extent of the targeting device based on a position of the probe.

15. The method of claim 14, wherein the probe comprises a radiopaque probe and the adjusting the distal extent of the targeting device comprises adjusting the distal extent based on a position of the probe viewed via an X-ray image.

16. The method of claim 14, wherein the probe comprises a hook, and the adjusting the distal extent of the targeting device comprises extending the hook to a posterior ligament.

17. The method of claim 9, further comprising placing a targeting pin on the bottom vertebra aligned with the second arm and inserting a spinal implant into the cavity in a direction defined by the targeting pin.

18. The method of claim 17, further comprising cutting a superior keel in the upper vertebra based on the position of the targeting pin.

19. The method of claim 18, further comprising cutting an inferior keel in the lower vertebra based on the position of the superior keel.

20. The method of claim 9, further comprising inserting a spinal implant into the cavity in a direction defined by the second arm.

21. The method of claim 9, further comprising positioning an intersection point of a longitudinal axis of the first arm and a longitudinal axis of the second arm at a mid-line of the spine and wherein the intersection point defines a target rotation center of a spinal implant.

22. The method of claim 21, further comprising inserting a spinal implant into the spinal cavity such that a rotation center of the spinal implant is located at the target rotation center.
